(19) United States
(12) Patent Application Publication
(10) Pub. No.: US 2004/0004007 A1
(43) Pub. Date: Jan. 8, 2004

(54) Apparatus, methods, and systems for cleaning and controlling bacteria growth, such as in fluid supply lines

(76) Inventors: John J. Orolin, West Linn, OR (US); Aaron R. Johnson, Hillsboro, OR (US); Kit G. Baldwin, West Linn, OR (US); Terry Allen Schmeiser, West Linn, OR (US); Jul Beth Johnson, (US); Christy J. Baldwin, (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/372,445

(22) Filed: Feb. 20, 2003

Related U.S. Application Data
(63) Continuation of application No. 10/205,473, filed on Jul. 24, 2002, now abandoned.
(60) Provisional application No. 60/308,277, filed on Jul. 26, 2001.
(60) Provisional application No. 60/355,401, filed on Feb. 8, 2002.

(51) Int. Cl. B08B 9/027

(52) U.S. Cl. 205/701; 205/742; 204/275.1; 134/22.11

(57) ABSTRACT

A noncaustic system for cleaning and controlling bacteria growth in fluid supply lines, containers, or on objects. The system includes an electrolytic cell generating aqueous solutions of supersaturated oxygen via electrolytic chemistry. The electrolyzed or treated water has both cleaning and antimicrobial effects when dispensed in fluid supply lines having beverage deposits, a layer or coating of bacteria, yeast, microorganisms or polysaccharide layers formed therein. The present invention further increases the germicidal activity of the above system toward microorganisms that may adhere and grow on the interior surfaces of the fluid supply line.

### BACKGROUND OF THE INVENTION

1. Field of the Invention

The present invention relates to cleaning and controlling bacteria growth and in particular, cleaning polysaccharides and other bacterial growth out of fluid supply lines using water treated by an electrolytic cell.

2. Description of the Related Art

In many industrial settings, fluid supply lines, containers or other surfaces become laden with yeast, bacteria, and other microorganisms that form polysaccharide layers that are hard to clean. Beverage and other fluid supply lines need to be systematically cleaned and sanitized to prevent the build-up of organic matter and microorganisms which can degrade product quality and cause health hazards. Additionally, these residues, layers, or deposits pose sanitary problems, especially when the fluid lines were those used to deliver liquids that humans drink or consume or otherwise intake. Some examples of these types of fluid lines include, but are not limited to, beer dispensing lines and water lines for other beverage equipment, dental hygiene water lines, dialysis and other medical supply lines, and condiment dispensing lines.

Caustic solutions, such as chemical cleaners and sanitizers, have typically been required to clean lines having bacterial growth. If improperly flushed or not flushed at all after cleaning, these caustic solutions have presented serious health problems when inadvertently consumed. Even if the lines had been properly rinsed after cleaning, chemical residues remained that impacted sensory characteristics as well as the general efficacy of these products. These caustics have been found to be dangerous to humans and posed an environmental hazard if not disposed of properly. Disposal of caustic solutions posed a significant economic and environmental burden. Also, caustic solutions needed to be heated to increase their cleaning effectiveness, which required extra steps in a cleaning process, including flushing the lines with ice water after cleaning. Heating of the lines also required that the fluid lines were constructed to endure heating and cooling cycles, often requiring more expensive material costs.

Thus, there is a significant need for a convenient, safe and environmentally harmless way to clean liquid supply lines that does not alter the product identity, in any setting where bacterial growth, and other microorganisms coat or deposit on the interior walls of fluid supply lines or form polysaccharides layers on the lines.

### BRIEF SUMMARY OF THE INVENTION

A system and method of cleaning and controlling bacteria growth, such as in fluid supply lines, containers, or on other surfaces is provided. One embodiment of the invention includes water treated by an electrolytic cell such that the treated water has high concentrations of dissolved oxygen. The electrolyzed or treated water has both cleaning and antimicrobial effects when dispensed in fluid supply lines having beverage deposits, a layer or coating of bacteria, yeast, microorganisms or polysaccharide layers formed therein. The present invention further increases the germicidal activity of the above system toward microorganisms that may adhere and grow on the interior surfaces of the fluid supply line.

According to one embodiment, the electrolytic cell is housed in an inline cartridge that can be directly fitted into a beverage line. Accordingly, the water can be delivered through the electrolytic cell in a single pass. Normal tap water can be passed directly through the electrolytic cell and then dispensed through the fluid lines being cleaned. In some embodiments, pressure transients can be induced in the fluid lines being cleaned to cause dissolved oxygen to rapidly come out of solution forming bubbles. The bubble formation can assists in breaking up polysaccharide layers.

According to another embodiment, water is transferred from a reservoir to an electrolytic cell to generate oxygen and elevate dissolved oxygen concentration in the water prior to passing the treated water through the fluid supply lines. Water can be re-circulating from the reservoir through the electrolytic cell for a period of time to build up the amount of dissolved oxygen in the water. Once the water is pre-charged with dissolved oxygen, the treated water is pumped through the lines to be cleaned until all the pre-charged water is dispensed. Pressure transients can also be induced when using a single pass to assist in cleaning.

In yet another embodiment, a cleaning system including a reservoir for holding a volume of water is connected to the electrolytic cell. A pump is coupled to the reservoir to deliver water from the reservoir to the electrolytic cell. An outlet valve is disposed upstream of an outlet port through which water is dispensed to the fluid supply lines to be treated. The outlet valve can be used to selectively dispense the water from the cleaning system. In one embodiment, a pre-charge valve is fluidly coupled to the electrolytic cell and the reservoir for controlling water flow from the electrolytic cell back to the reservoir. When the pre-charge valve is opened, water can be re-circulated between the electrolytic cell and the reservoir. In another embodiment, a flow activated switch is coupled to the electrolytic cell for closing a power supply circuit path to the electrolytic cell only when the electrolytic cell is flooded and water flow is established out of the electrolytic cell.

### BRIEF DESCRIPTION OF THE SEVERAL VIEWS OF THE DRAWING(S)

FIG. 1 is a process and instrumentation diagram in accordance with one embodiment of the cleaning system of the present invention.

FIG. 2 is a block diagram of one embodiment of an enclosure for the present invention, including a block diagram of the control panel in accordance with principles of the present invention.

FIG. 3 is a graph illustrating test results demonstrating the efficacy of the treated water in removing microbial biofilms from fluid supply lines in accordance with the principles of the present invention.

FIG. 4 is a schematic view of the system of Experiment #1 in accordance with the principles of the present invention.

### DETAILED DESCRIPTION OF THE INVENTION

The following references are incorporated herein by reference in their entireties: U.S. Pat. No. 6,171,469, filed Oct. 31, 1996; U.S. Pat. No. 5,728,287, filed Oct. 31, 1996; U.S. Pat. No. 5,911,870, filed Apr. 4, 1997; U.S. Pat. No. 6,296,756, filed Sep. 9, 1999; U.S. Pat. No. 6,332,972, filed Dec. 17, 1999; U.S. patent application Ser. No. 09/575,727, filed Aug. 4, 2000; and, U.S. patent application Ser. No. 09/637,755, filed Mar. 1, 2000.

In the following description, certain specific details are set forth in order to provide a thorough understanding of various embodiments of the invention. However, upon reviewing this disclosure one skilled in the art will understand that the invention may be practiced without many of these details. In other instances, well known structures associated with pumps, tanks, switches, and various process instruments associated with liquid transfer, pressurizing, and processing systems have not been described in detail to avoid unnecessarily obscuring the descriptions of the embodiments of the invention.

Some specific embodiments of the invention set forth below are described and illustrated as applied to cleaning supply lines used to deliver fluid, for example, beer, such as lines used to dispense beer, or any other fluid lines ancillary thereto. Also, the present invention can be applied to fluid lines used to transfer solutions during the manufacturing or processing of the fluid. However, as one skilled in the art will appreciate after reviewing this disclosure, the present invention has broad applicability to fulfill a wide variety cleaning needs for many types of surfaces, including the inside walls of any fluid lines and any other surface having bacteria, yeast, polysaccharide or microorganism residue, deposits or films.

Examples of fluid supply lines to which the present invention can be applied include but are not limited to the following: potable water supply lines; beer lines and other beverage dispensing lines and ancillary equipment including various distribution piping to transfer bulk products to market; dental rinse water dispensing equipment; dialysis and other medical supply lines; containers for organic matter; and condiment dispensing equipment. In such fluid lines bacteria or other microorganisms or polysaccharide films can pose sanitary problems and health risks, as the fluid lines contact fluids that are used orally or ingested by humans. The following description and Experiments illustrate that the cleaning system of the instant invention has high efficacy in cleaning and controlling bacteria in such lines without the use of caustic chemicals.

Significantly, the cleaning methods and apparatus of the invention do not require the use of caustics (commonly used to clean lines contaminated by bacterial growths as discussed in the Background) which can be hazardous to humans and environmentally unsafe themselves, as well as expensive to use. Additionally, no preheating of the cleaning water or solution is needed in the present system, which is often required when cleaning with caustics. This, in turn, also eliminates any need to flush lines with ice water after cleaning, an additional and inconvenient step required in the prior art. The present invention thus provides effective cleaning while being easier to use, reduces or eliminates environmental hazards by avoiding the use of caustic, and provides improved cleaning economics.

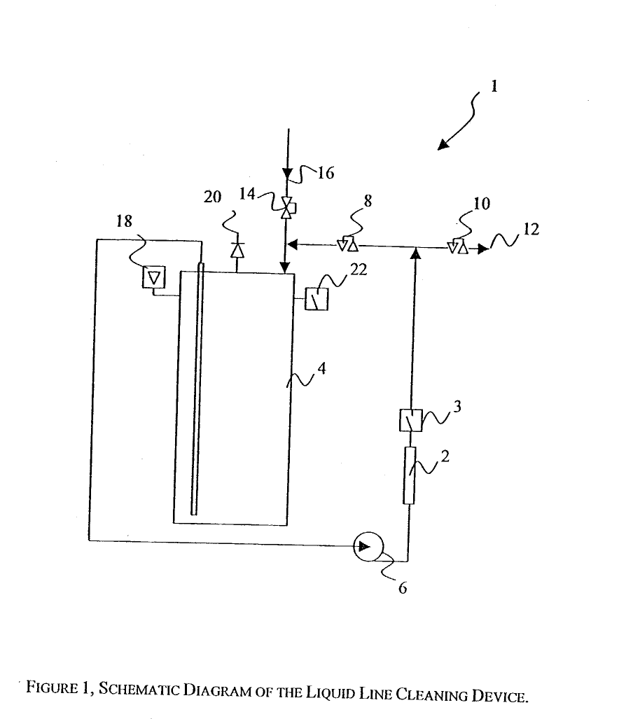

FIG. 1 illustrates one embodiment of a cleaning system 1 of the present invention as applied to beer lines. In overview, the cleaning system includes various units, including an electrolytic cell 2, a reservoir tank 4, and a pump 6. Water (or an aqueous solution) in the reservoir tank 4 can be pumped through the electrolytic cell 2 using the pump 6. Within the electrolytic cell 2, an anode and electrode drive an electrolysis reaction to disassociate oxygen from water molecules thereby elevating the concentration of dissolved oxygen in the water. Some electrolytic cells and associated systems set forth in references incorporated supra can be employed in conjunction with the present invention, in manners one skilled in the art will appreciated upon reviewing this disclosure, to produce high levels of stable dissolved oxygen. The electrolyzed, or treated, water (with high oxygen content) is then discharged into lines, containers or on objects that are to be cleaned.

The reservoir tank 4 has an inlet line with a fill valve 14 and inlet port 16. The inlet port 16 is configured to be easily connectable to a tap water source for filling the reservoir tank 4. Another fluid line connects an outlet of the reservoir tank 4 to a suction side of the pump 6, with a discharge side of the pump 6 being connected to an inlet of the electrolytic cell 2, such that liquid can be pumped from the reservoir tank 4 to the electrolytic cell 2. The pump 6 can be a positive displacement pump, but in some embodiments, can also be a centrifugal type pump, or a pump having centrifugal characteristics.

In the illustrative embodiment, the electrolytic cell 2 has an outlet that is fluidly connected to both a pre-charge valve 8 and an outlet valve 10. The pre-charge valve 8 is, in turn, connected back to the inlet line of the reservoir tank, downstream of the fill valve 14. This allows electrolyzed water passing through the pre-charge valve 8 to be directed back to the reservoir tank 4. The outlet valve 10 is used for selectively discharging electrolyzed water from the cleaning system 1 and can be formed with an outlet port 12 or be fluidly connected to an outlet port 12. The outlet port 12 is configured to be easily connectable to beer lines for discharging electrolyzed water from the cleaning system 1 into the beer lines.

In the illustrated embodiment in FIG. 1, the fill valve 14, pre-charge valve 8, and outlet valve 10 are each solenoid valves, capable of being remotely actuated between open positions, to allow water to flow through the valves, and closed positions, to stop flow-through. Alternatively, ball valves, check valves, or other known valves may be used and may be remotely or manually actuated. Therefore, the system can be controlled manually with mechanical valves and mechanical switches to turn on or off the pump and electrolytic cell, or the system can also be controlled electrically via mechanical switches for all electrical components. The device can also be controlled via electronics in an automatic fashion.

In the illustrated embodiment, the reservoir tank 4 is also provided with an exhaust valve 18 for exhausting gas from the tank so that it does not build up pressure during filling, as well as a relief valve 20, for pressure relief when the exhaust valve is closed (e.g. emergency pressure relief). The exhaust valve 18 can be a float valve, or be actuated by a float within the reservoir tank 4 to close the exhaust valve when the liquid level is full in the reservoir tank 4 and to open the exhaust valve 18 when the liquid level drops. This prevents water in the reservoir tank from overflowing the exhaust valve 18. Furthermore, the float, or any other type of level or liquid head sensing device, can be provided to trip a level switch 22 coupled to a level indicating device (such as the FULL INDICATOR LIGHT 38 described below) to indicate a liquid level in the reservoir tank 4. In one embodiment, the level switch 22 can be an automatic level control switch coupled to the fill valve 14 to close it when the liquid level is full in the reservoir tank 4, or to open it when the liquid level drops. This can avoid overfilling or over pressurizing the reservoir tank 4 while maintaining a predetermined level in the tank until the automatic level control switch is shut off.

In yet another embodiment, in addition to the level control switch 22, a cell-ready switch 3 can be provided for the electrolytic cell 2. The cell-ready switch 3 can be configured to break a power supply circuit path to the electrolytic cell when it is opened, and to complete the power circuit thereto when closed. Also, the cell-ready switch 3 can coupled to a flow-sensing device which is disposed in the fluid line downstream of the electrolytic cell 2. The flow-sensing device can be configured to close the cell-ready switch 3, and allow power to be supplied to the electrolytic cell 2, only upon sensing water flow out of the cell. This allows the electrolytic cell to flood, or fill, before power will be supplied to the electrodes of the cell.

In some embodiments of the cleaning system 1, a device can be provided to indicate when the fluid lines have been adequately flushed (during the dispensing phase). For example, a sensing device can be provided to sense purity of water that is flushed through the fluid lines. The sensing device can include quantitative or qualitative devices to gauge water purity with respect to the types of layers, deposits, or films being removed from the lines. This can include, for example, without limitation, turbidity meters to detect turbidity of the water or biological sensing devices to directly detect the presence of microorganisms in water flushed from the lines being cleaned. The sensing devices can be in-line sensing devices, or off-line requiring a sample to be draw (e.g. bioluminescence tests). The sensing devices can also be integral to the cleaning system 1 or separate. Alternatively, a timing system can also be employed to control flushing time through the fluid lines being cleaned. The timing system could have preset times that are empirically determined to be effective flushing or dispensing times and could be based on the type of line being flushed or the severity of buildup in the lines. Any of the preset times may be selected. In yet another embodiment, a run time can be specified and the timing can be manually operated.

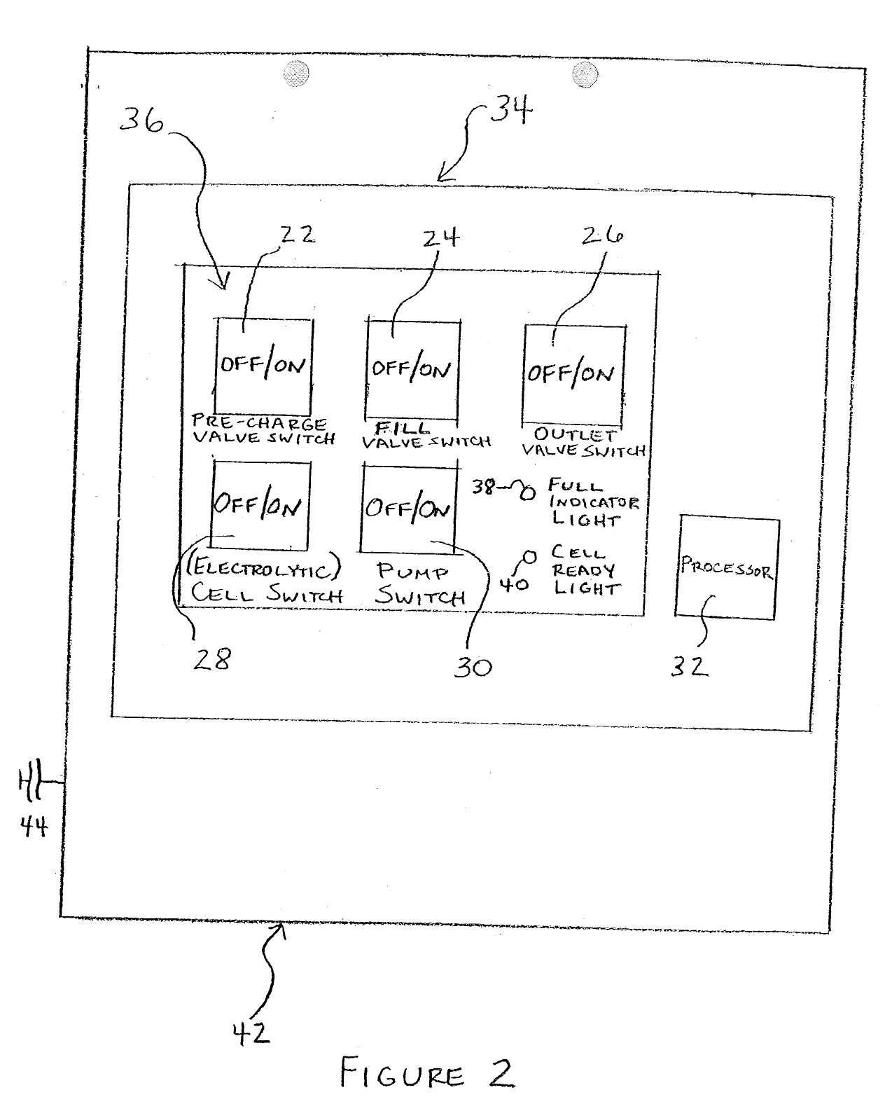

The cleaning system 1 can also have a control center 34, with a control panel 36, as illustrated in FIG. 2. In one embodiment, the control panel 36 includes manual valve switches (i.e. pre-charge valve switch 22, fill valve switch 24, and outlet valve switch 26) for remotely closing or opening each of the solenoid valves, as well as an electrolytic cell switch 28 and a pump switch 30, for supplying or shutting off power to the those units respectively.

In some embodiments, the entire cleaning system 1 is enclosed within a single enclosure with the control center 34 being coupled to, or formed within, the enclosure and the control panel 36 being formed or installed on a top or side portion of the enclosure.

Some embodiments of the present invention comprise at least two modes of operation, or cleaning cycles: a pre-charge mode and a single-pass mode. In one embodiment of the pre-charge mode, the cleaning system 1 is connected to a normal tap water source (city water) via the inlet port 16, and the outlet port 12 is connected to the lines to be cleaned, such as beer lines, as illustrated in FIG. 1. An outlet end of the beer lines to be cleaned is disposed in a sanitary drain. Tap water is then supplied to the reservoir tank 4, through the fill valve 16. The pre-charge valve 8 is opened and the outlet valve 10 is closed and once the reservoir tank 4 is full, or has sufficient level for providing adequate water head to the pump 6, the pump is started. Water is then circulated from the reservoir tank, through the electrolytic cell 2 (to which power is being supplied), and back to the reservoir tank 4 through the open pre-charge valve 8. This is continued for a period of time sufficient to build up a desired concentration of dissolved oxygen in the water. Once the water is pre-charged with dissolved oxygen to a desired level of oxygen concentration, the water is pumped through the lines to be cleaned, by opening the outlet valve 10 and closing the pre-charge valve 8. This discharges, or dispenses, the pre-charged electrolyzed water to the beer lines, to effectively clean the beer lines.

In some embodiments pressure transients can be induced in the beer lines being cleaned during the discharge phase by cycling the outlet valve 10 open and closed while discharging the electrolyzed water to the beer lines. During the low pressure transients, dissolved oxygen can rapidly come out of solution causing bubbles. This action in turn assists in breaking up polysaccharide layers in the tubing being cleaned.

In still further embodiments, additives such as sodium chloride (normal table salt) can be added to the water to create chlorine during the circulation phase of the pre-charge mode. Adding sodium chloride, however, is not essential to effective cleaning with the cleaning system 1. Alternative additives can be added to the fluid to produce varying effects.

When the cleaning system 1 is operated in the single-pass mode, water can be pumped from the reservoir tank 4 through the electrolytic cell 2 to be dispensed, without re-circulating the water through the electrolytic cell 2 (i.e. no pre-charge phase). This can comprise closing the pre-charge valve 8 and opening the outlet valve 10 before starting the pump 6, to directly pump water through the electrolytic cell 2 out to the beer lines.

In some embodiments of the single-pass mode, pressure transients can be induced, like in the pre-charge mode dispensing phase, by cycling the outlet valve 10 between closed and open positions. Again, this can assist in cleaning the beer lines by causing low pressure transients wherein dissolved oxygen rapidly bubbles out of the electrolyzed water to scrub the beer lines. Without being bound by theory, it is also noted that pressure transients, regardless of mode of operation, can also be induced with other valves besides the outlet valve 10. For example, a separate pressuring transient inducing valve can be installed in another location in the lines of the cleaning system 1. Also, in some embodiments (such as those wherein the pump 6 is a centrifugal pump) the pre-charge valve 8 can be cycled while the outlet valve 10 is left open in a hybrid pre-charge/single-pass mode. In these hybrid embodiments, cycling the pre-charge valve 8 can cycle backpressure on the discharge side of the pump 6, thereby causing pressure transients in the lines, including the lines being cleaned. In addition, a portion of the water would be re-circulated back through the electrolytic cell 2 while another portion is directly dispensed.

One embodiment of operating procedures for the pre-charge mode comprises the following steps:

1. Connect cold tap water to the water inlet port 16. Connect the outlet port 12 to the beer lines and daisy chain the beer lines together. The output of the last beer line in the chain should be directed to a sanitary drain.

2. Check to see that all switches 22, 24, 26, 28, and 30 on the control panel 36, of the Cleaning System 1 are in the OFF position. Connect the 110 v power cord 44. Ensure that the tap water is turned ON.

3. Turn ON the FILL VALVE SWITCH 24. Wait for the FULL INDICATOR LIGHT 38 to illuminate.

4. Turn ON the PRECHARGE VALVE SWITCH 22.

5. Turn ON the PUMP SWITCH 30. Wait for the CELL READY LIGHT 40 to illuminate.

6. Turn ON the CELL SWITCH 28.

7. Turn OFF the FILL VALVE SWITCH 24. Wait 15 minutes.

8. Turn ON the OUTLET VALVE SWITCH 26.

9. Turn OFF the PRECHARGE VALVE SWITCH 22. Watch the CELL READY LIGHT 40. (It should take about 9½ minutes to empty the tank and for flow to stop.)

10. When the CELL READY LIGHT 40 goes out, turn OFF the CELL SWITCH 28.

11. Turn OFF the PUMP SWITCH 30.

12. Turn OFF the OUTLET VALVE SWITCH 26.

One embodiment of operating procedures for the single-pass mode comprises the following steps:

1. Connect cold tap water to the water inlet port 16. Connect the outlet port 12 to the beer lines and daisy chain the beer lines together. The output of the last beer line in the chain should be directed to a sanitary drain.

2. Check to see that all switches 22, 24, 26, 28, and 30, on the control panel 36 of the cleaning system 1 are in the OFF position. Connect the 110v power cord 44. Ensure that the tap water is turned ON.

3. Turn ON the FILL VALVE SWITCH 24. Wait for the FULL INDICATOR LIGHT 38 to illuminate.

4. Turn ON the OUTLET VALVE SWITCH 26.

5. Turn ON the PUMP SWITCH 30. Wait for the CELL READY LIGHT 40 to illuminate.

6. Turn ON the CELL SWITCH 28. Wait 10 to 15 minutes.

7. Turn OFF the CELL SWITCH 28.

8. Turn OFF the PUMP SWITCH 30.

9. Turn OFF the OUTLET VAVLE SWITCH 26 and FILL VALVE SWITCH 24.

In some embodiments, the cleaning system 1 may be flushed after use. One example of operation of the cleaning system during a flushing cycle can be accomplished via the following steps:

1. Connect cold tap water to the water inlet port 16. Connect the outlet port 12 to the beer lines and daisy chain the beer lines together. The output of the last beer line in the chain should be directed to a sanitary drain.

2. Check to see that all switches 22, 24, 26, 28, and 30 on the control panel 36 of the cleaning system 1 are in the OFF position. Connect the 110v power cord 44. Ensure that the tap water is turned ON.

3. Turn ON the FILL VALVE SWITCH 24. Wait for the FULL INDICATOR LIGHT 38 to illuminate.

4. Turn ON the OUTLET VALVE SWITCH 26.

5. Turn ON the PUMP SWITCH 30. See that the CELL READY LIGHT 40 is illuminated. Wait 5 to 10 minutes.

6. Turn OFF the FILL VALVE SWITCH 24. Watch the CELL READY LIGHT 40. (It should take about 9½ minutes to empty the tank and for flow to stop.)

7. Turn OFF the PUMP SWITCH 30.

8. Turn OFF the OUTLET VALVE SWITCH 26 and FILL VALVE SWITCH 24.

The cleaning system 1 can be configured to be manually controlled so that each step above is manually carried out as described. Also, the valves and switches could be manually controlled valves (i.e. not remotely controlled with switches). In addition, the cleaning system 1 can be automated with many of the operating steps being interlocked with switches and timers, or being controlled by a processor 32 in the control center.

According to the principles of the present invention, the treated water can be used in a variety of ways, including but not limited to: to clean bacteria, yeast, polysaccharides films, and other microorganisms from beer dispensing lines and ancillary equipment; to clean dental rinse water dispensing equipment of bacteria and other micro-organisms and polysaccharide films; to clean and control bacteria in dialysis and other medical fluid supply lines; to clean beverage dispensing equipment fluid lines and control bacteria and other micro-organisms and poly-sacaride films; to clean condiment dispensing equipment of bacteria and other micro-organisms and poly-sacaride films; to provide antimicrobial effects on the surfaces treated. To clean industrial equipment fluid lines where bacteria or other microorganisms or poly-sacaride films can pose sanitary problems.

There are at least two methods of operation for the cleaning system, the pre-charge method and the single-pass method. In the pre-charge method, normal tap water is drawn into a reservoir and then circulated through an electrolytic cell via appropriate plumbing and a pump for a period of time to build up the amount of dissolved oxygen in the water. It is optional to add sodium chloride to the water to create chlorine during this process; however, it is not essential to the method of cleaning. Once the water is pre-charged with dissolved oxygen, the water is pumped through the lines to be cleaned until all the pre-charged water is dispensed. An optional method of employing pressure transients to the lines is to cycle a solenoid valve open and closed during the dispensing operation. During the low pressure transients, dissolved oxygen can rapidly come out of solution causing bubbles. This action in turn assists in breaking up poly-sacaride layers in the tubing being cleaned.

In the single pass method, normal tap water is passed directly through the electrolytic cell and then through the lines or onto the surface to be cleaned via appropriate plumbing and a pump. Again, the induction of pressure transients is an optional operation to assist in the cleaning.

Thus, beer dispensing lines, dental hygiene water lines, medical lines, water supply lines, beverage dispensing equipment lines, condiment dispensing lines, other industrial process lines and the like, are often plagued by bacterial growth and other micro-organisms that form poly-sacaride layers and are very difficult to clean. Caustic solutions are usually required to clean this type of bacterial growth. These caustics are dangerous to humans and pose an environmental hazard if not disposed of properly. The present invention offers a very effective method of cleaning and controlling these bacterial growths without the use of caustics. The process is therefore more economical than using caustics. Further, often the caustic solutions are heated to make their use more effective. In beverage lines, this then requires that the lines be flushed with ice water before the beverage can be re-introduced into the lines. The present invention does not require the water to be heated and thus flushing with ice water is unnecessary. The process of the present invention is usually faster than the caustic cleaning process and thus less time must be invested by the personnel that conduct the cleaning.

In the Experiment below, the cleaning system includes the following components: 1) a solenoid valve to control the flow of tap water into the system (referred to as the fill valve), 2) a solenoid valve to control the path of water through the system (referred to as the precharge valve), 3) a solenoid valve to control the flow of water out of the system (referred to as the outlet valve, 4) a gas vent that allows gases to exit from the reservoir tank but does not allow water to exit from the tank (this distinction is accomplished by a float valve), 5) a pressure relief valve, 6) a level switch that electrically closes when the tank is full, 7) a reservoir tank, 8) a flow switch that electrically closes when water is flowing through it, 9) the electrolytic cell, and 10) a pump.

The present invention is not limited by the following Experiments. The Experiments are shown for illustrative purposes only.

### Experiment #1: Inline Bacteria Cleaning System

Overview

The present experiment includes an electrolytic cell housed in an inline cartridge adapted to be directly filtered into a beverage line.

This test system includes an oxygen-generating cartridge configured in a closed loop of tubing to provide a recirculating effect. Within the loop there are fittings to accommodate test pieces of tubing that have been intentionally allowed to accumulate organic material and/or microorganisms on the interior surfaces. In addition there are sample ports within the loop to allow the removal of aqueous samples. The loop can also have a T connector to allow a water rinse cycle after treatment. To simulate beverage line soiling and microbial contamination, the tubing samples are exposed to draft beer and allow the deposition of organic matter, which is mostly beer protein, onto the surfaces. The tubing surfaces become colonized by typical beer spoilage microorganisms such as Lactobacillus, Pediococcus and Acetobacter. The cleaning ability of the present invention is evaluated by measuring the amount of organic matter before and after treatments. This may be done with analytical protein assays as well as visual staining procedures. Antimicrobial activity is assayed by performing microbial plate counts before and after treatments. In addition, ATP bioluminescence measurements may be used as a qualitative assessment of cleaning.

Beverage Line Cleaning Test System Operation Protocol

In operation, the beverage line cleaning system of the present invention was shown to be effective in reducing bacterial counts, and further produced a cleansing effect on contaminated beer lines. Operation of the electrolytic cell produces high levels (>20 ppm) of dissolved oxygen and converts a high proportion of any chloride ion present to hypochlorite ion. The combined presence of high dissolved oxygen and low levels of hypochlorite ions combined with the relatively strong electromagnetic fields produced within the cell, served to reduce bacterial levels.

The present system circulates tap water treated by the electrolytic cell though contaminated beer lines along with periodic rinsing to reduce undesirable bacterial levels and remove plaques and mold.

One embodiment of the present invention includes the operation of an electrolytic cell and pump system using tap water circulating through tubing purposely contaminated with selected bacteria. The system will be tested using two cells, one of which is designed to suppress hypochlorite production. Measured responses will be dissolved oxygen (DO), temperature, pH, hypochlorite (free chlorine), and residual bacterial levels, both in the rinse water and on tubing walls. The system will be operated for short periods of time then purged and refilled with fresh tap water.

Various test equipment is used to verify performance and configure the system. For example, one test station is composed of an SPS-1 EC400 electrolytic cell, DC constant current power supply, pressure gage, gas relief valve, 0-5 gpm flow meter, and Micro Pump variable speed gear pump; SPS-1 EC626 electrolytic cell; DO meter and probe; pH meter; chlorine test strips; chloride test strips; conductivity meter; tubing; bacterial cultures; and a timer.

Narrative of Procedure: One procedure for testing the efficacy of the system is as follows.

Connect tubing to be tested to the test system. Tubing should be placed above system to allow for complete draining. Slowly fill the Test System by opening ball valve slightly. Take care to minimize bubble formation during filling. Take a one-liter sample of the tap water for analysis. Turn voltage control knobs on the DC power supply fully clockwise, and turn current control knobs fully counter clockwise. Turn Micro Pump toggle switch to controller position, and increase speed control to produce 0.5 gpm of flow. Turn DC power supply on and adjust current control knobs to 5.0A. Start timer and run system for 10 minutes. Stop system after 10 minutes and drain system. Open fill ball valve and flush system for 30 seconds. Refill system as in 9.2, and operate system for an additional 10 minutes. Stop system and take sample for analysis. After all tests are completed using the SPS-1 EC400 electrolytic cell, replace the cell with the SPS-1 EC626 electrolytic cell. Repeat all tests with the SPS-1 EC626 electrolytic cell. Analyze all samples per analysis plan.

Outline of Procedure

1. Background

1.1 Beverage system lines, i.e., soda fountain and draft beer, need to be periodically cleaned to maintain control of bacteria growth.

1.2 The ⅜″ flexible product delivery lines are normally disinfected weekly by contracted personnel.

1.3 Bacteria, molds, and yeast grow in these lines. These growths can cause odor, taste, and health problems if not addressed in a timely manner.

2. Purpose

2.1 The purpose of this procedure is to measure the effects of water treated by an electrolytic cell on bacteria in contaminated beverage lines.

3. Equipment

3.1 Three (3) separate six (6) foot lengths of ⅜″ beverage lines

A. One Beverage line from a draft beer dispensing system.

B. Two (2)-beverage lines from a soda fountain system.

Each line was sealed and contained approx. 100 ml of liquid within the tubing.

3.2. Non-Chemical bacterial cleaning system consisting of the following:

A. Electrolytic cell.

B. Mixture Chamber.

C. Recirculation Pump.

D. Gas Relief Valve.

E. 24VDC Power Supply with wire connection to the cell.

F. Reservoir for addition of water.

See FIG. 1.

4. Initial

4.1 Insure all meters and probes are operational, and have been calibrated prior to carryout measurements

4.2 Turn pH and DO meters on 30 minutes prior to testing.

4.3. Assemble and setup test station and connect power supply.

5. Setup

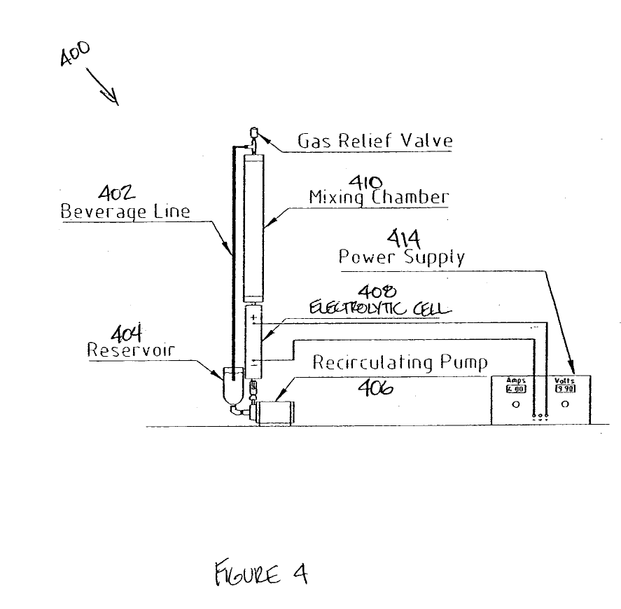

5.1 Refer to FIG. 4 for the Test setup. The system 400 includes a beverage line 402 fluidly connected to an optional reservoir 404. The reservoir 404 is fluidly coupled with a recirculating pump 406. The recirculating pump 406 fluidly couples with and directs water through an electrolytic cell 408 and mixing chamber 410. A gas relief valve 412 is provided to off gas any gaseous byproduct of the electrolysis process. A power supply 414 is electrically connected to the electrolytic cell 408. Alternatively, the beverage line 402 can be fluidly connected to bypass piping and the reservoir 404 can be eliminated. The bypass piping can direct the water through the electrolytic cell 408 for treatment prior to passing the treated water through the beverage line 402.

6. Procedure

6.1 Attach the Beer dispensing line to the cleaning system.

6.2 Allow the liquid contained within the sealed line to siphon into the reservoir and add a quantity of 500 ml of tap water.

6.3 Apply power to the pump to allow circulation of this mixture within the system and do not apply power to the cell.

6.4 Turn off the pump.

6.5 Take a sample of this new homogenous mixture and label “Initial untreated sample.”

6.6. Reapply power to the pump to start circulation.

6.7 Turn on the power supply for the cell and adjust it in a constant current mode to provide 4.0 Amps to the cell for oxygen production.

6.8 Allow this system to run for 10 minutes.

6.9 Turn off all power to the cell and pump.

6.10 Take a sample of this new oxygen enriched homogenous mixture and label “Treated sample.”

6.11 Drain the entire system.

6.12 Fill the system with tap water.

6.13 Reapply power to the pump only.

6.14 Allow the tap water to circulate for 5 minutes.

6.15 Turn off power to the pump.

6.16 Take a sample of this rinsed system and label “Rinsed Tube Sample.”

6.17 Drain the system.

6.18 Repeat steps 6.1-6.17 for the other beverage lines to be tested.

Observations

The Table of results for this test is listed below:

TABLE #1

| | | |
|---|---|---|
| Untreated | Treated | Rinse |
| Sample | Sample | Sample |

Beer Line Samples

| | | | |
|---|---|---|---|
| Dissolved Oxygen (ppm) | 8.7 | 13.9 | 9.1 |
| Conductance (ᵤS) | 150 | 150 | 140 |
| PH | 6.8 | 6.8 | 6.9 |
| Free Chlorine (ppm) | <0.5 | 3 | <0.5 |
| Total chlorine (ppm) | <0.5 | 3.5 | <0.5 |
| Alkalinity | 80 | 80 | 80 |
| Temperature (° C.) | 17.2 | 22.1 | 16.2 |
| Hardness | 140 | 140 | 150 |
| Molds | <1 | <1 | <1 |
| Yeast | >6,000,000 | 140 | <1 |
| HPC | >6,000,000 | 95 | <1 |
| Amperage | N/A | 4.00 | N/A |
| Voltage | N/A | 9.90 | N/A |

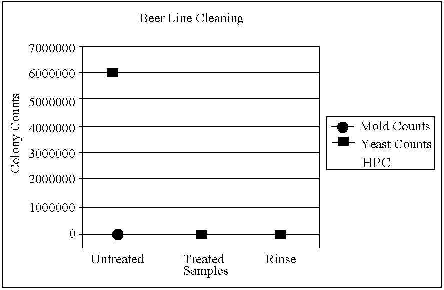

Soda Line Samples

| | | | |
|---|---|---|---|
| Dissolved Oxygen (ppm) | 6.3 | 14.00 | 8.9 |
| Conductance (ᵤS) | 2700 | 2600 | 220 |
| PH | 8.4 | 8.4 | 7.8 |
| Free Chlorine (ppm) | <0.5 | 3.5 | <0.5 |
| Total chlorine (ppm) | <0.5 | 4.0 | <0.5 |
| Alkalinity | 180 | 180 | 180 |
| Temperature (° C.) | 17.2 | 22.0 | 16.7 |
| Hardness | 350 | 350 | 170 |
| Molds | 100 | <1 | <1 |
| Yeast | <1 | <1 | <1 |
| HPC | 10,000 | <1 | <1 |
| Amperage | N/A | 4.00 | N/A |
| Voltage | N/A | 5.00 | N/A |

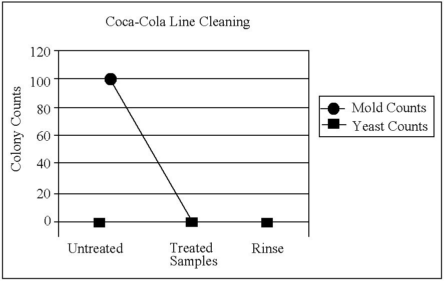

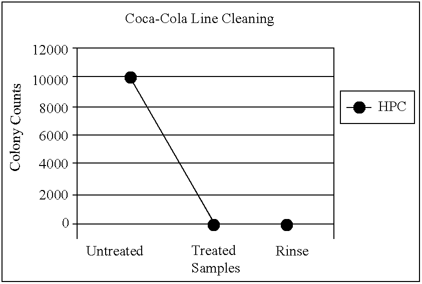

Conclusion

The goal set in the cleaning of the beverage lines was a 4 Log reduction in bacterial populations, mold, and yeast colonies after tap water rinsing. The studies of both the draft beer delivery and soda delivery tubing showed this level of reduction was achieved. Normal tap water contains a level of chloride that can be measured. The use of the SPS-1 200 electrolytic cell will enhance the conversion of chloride into chlorine. This conversion is further enhanced due to the repeated passes in a circulation environment. If some beverage tubing is more troublesome in bacterial destruction, longer treatment times or more frequent treatments could be applied.

### Experiment #2

Objectives

The Object of this Experiment is to evaluate the efficacy of various configurations. One test system consists of a direct throughput system and another includes recirculation through the electrolytic cell, which is then followed by direct throughput. These systems will be tested in various configurations to determine the efficacy in eliminating spoilage microorganisms from beverage transport tubing. Also an evaluation will be undertaken to determine whether the inclusion of salt to the system (to generate chlorine) increased the antimicrobial efficacy.

Materials and Methods

The following protocol was employed:

SPS-1 Electrolytic Cell Operation Procedure for the Beverage Line Cleaning

1. Turn on tap water connected to the electrolytic cell.

2. Open ball valve on the incoming water supply.

3. Turn three way valve #1 to the black mark. Adjust this valve to maintain 0.5 gal/min.

4. Turn three way valve #2 to the dispense mode.

5. System will fill with water (remove all large bubbles) and water will be draining.

6. Make sure system has no leads and is flowing properly.

7. turn on power supply to 5 amps.

8. Begin testing procedure for 10 minutes etc.

Operation Procedure for Re-Circulation Tank System

1. Repeat steps 1-6 above.

2. Turn off ball valve.

3. Fill tank with tap water (about 5 gallons).

4. Turn valve #1 to dispense.

5. Turn valve #2 to sanitize.

6. Turn on pump.

7. Set flow to 0.5 gal/min with valve #1.

8. Turn on power supply to 5 amps.

9. Let the tap water re-circulate for 15 minutes.

10. To activate the cleaning system, turn valve #2 to dispense.

11. Maintain 0.5 gal/min with valve #1.

12. The tank will drain during the cleaning cycle.

Test Protocols

Test #1

A place will be provided to attach the contaminated polypropylene tube to the apparatus. The single pass system was activated for 10 minutes at 5 amps. Before and after samples will be taken of the tubing for bacterial and yeast reductions.

Test #2

The single pass system will be activated for 5 minutes at 5 amps. Before and after samples will be taken of the tubing for bacterial and yeast reductions.

Test #3

The single pass system will be activated for 5 minutes at 5 amps. The solenoid will be pulsed approximately 20 times and minute for the 5 minute testing period. Before and after samples will be taken of the tubing for bacterial and yeast reductions.

Test #4

The single pass system will first charge a 5 gallon tank for 10 minutes using a re-circulation setup. The single pass system will be activated for 10 minutes at 5 amps using the water from the pre-charged tank. Before and after samples will be taken of the tubing for bacterial and yeast reductions.

Test #5

The single pass system will first charge a 5 gallon tank for 10 minutes using a re-circulation setup. The single pass system will be activated for 5 minutes at 5 amps using the water from the pre-charged tank. Before and after samples will be taken of the tubing for bacterial and yeast reductions.

Test #6

The single pass system will first charge a 5 gallon tank for 10 minutes using a re-circulation setup with 2 grams of table salt added to the water. The single pass system will be activated for 5 minutes at 5 amps using the water from the pre-charged tank. Before and after samples will be taken of the tubing for bacterial and yeast reductions.

Comments

A mixture of beer spoilage microorganisms was used as an inoculum to grow biofilms upon the surfaces of a ⅜″ i.d. clear tygon type tubing. A section of the biofilm containing tubing was inserted into each system prior to the test run. A control consisted of the biofilm containing tubing directly assayed for microbial populations without under going any treatment. After each test run, the tubing was removed and assayed for microbial populations using plate count agar which will give us the total aerobic plate count. ATP and well as chlorine measurements were taken. Each test run was performed in duplicate and were designated: 1a, 1b, 2a, 2b, 3a, 3b, 4a, 4b, 5a, 5b, 6a, and 6b.

A separate experiment was performed to evaluate if chlorine production was necessary for the antimicrobial activity of electrolyzed water. We used the Test # 5 system protocol as described above but modified as follows. We filled the 5 gallon reservoir with deionized water that had a conductivity of less than of 2.0 microsiemens. To that was added sodium chloride (2 grams) which gave a conductivity of 187.8 microsiemens. This water when circulating through the system gave us 6.9 volts when the amperage was set at 5.0 amps. We then fitted the biofilm containing tubing and ran 2 separate tests to determine the antimicrobial effectiveness. In this system, significant amounts of chlorine (>10 ppm) were generated. We then again filled the 5 gallon reservoir with deionized water but this time we added sodium citrate to an amount (3.7 grams) that gave us the same conductivity value as the sodium chloride did. When circulating the system at 5.0 amps we had a voltage reading of 7.0. thus, we have equivalent systems in terms of conductivity and sodium ion with the only difference being that with the sodium chloride we will generate chlorine but with the sodium citrate we will not. These tests were designated 7a, 7b (sodium chloride) and 8a, 8b (sodium citrate).

Results

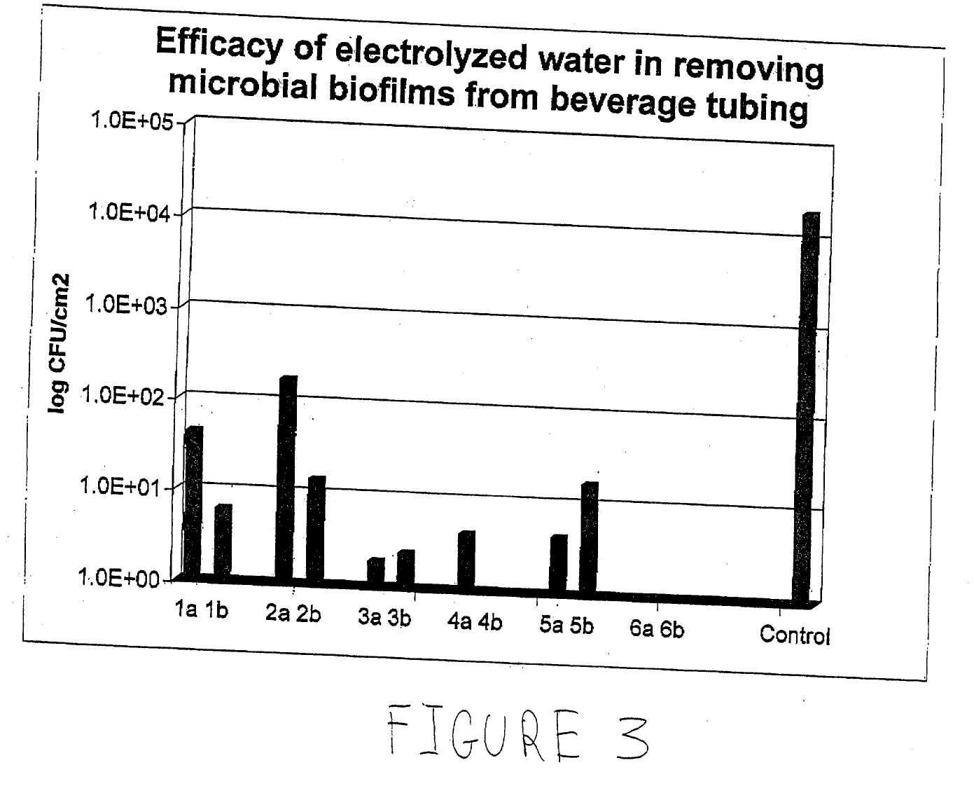

As shown in the attached graph (FIG. 3) all treatments were effective in removing bacterial populations (>99%) from the beverage transport tubing as compared to the non-treated control tubing. Test system #6, which included sodium chloride, showed all microorganisms were eliminated. In general the recirculating systems (#4,5 and 6) were more effective than the simple thru passing systems (#1 and 2). The solenoid—pulse system (#3) appeared to increase the effectiveness of the single pass systems. In general the ATP data correlated with the plate count data. Residual chlorine for all tests was 1 ppm or less with the exception of #6 which was greater than 10 ppm.

The results for tests 7a, 7b, 8a and 8b were not graphed because there were no surviving microorganisms from any of the treatments. The control treatment had 6.1×10⁵ CFU/ml. These results suggest that chlorine generation is not a prerequisite for antimicrobial activity. Residual chlorine for the # 8 tests was 1 ppm or less and for #7 was greater than 10 ppm. Since the conductivity was the same for both the #7 and # 8 treatments, it may be that the rate of electrolysis is the most important factor for antimicrobial activity.

The above description of illustrated embodiments and experiments of the invention are not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other fluid supply line systems, not necessarily the beverage supply lines described above.

The various embodiments and experiments described above can be combined to provide further embodiments. All of the above U.S. patents and applications are incorporated by reference. Aspects of the invention can be modified, if necessary, to employ the systems, supply lines, electrolytic cells and concepts of the various patents and applications described above to provide yet further embodiments of the invention.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of cleaning a surface of bacteria comprising:
circulating water through an electrolytic cell to produce treated water; and
passing the treated water across a surface to reduce the bacteria count on the surface.

2. The method of claim 1 further comprising re-circulating the water through the electrolytic cell to further treat the water prior to passing the water across the surface.

3. The method of claim 2 wherein re-circulating the water comprises delivering the water from the electrolytic cell to a reservoir to be mixed with water in the reservoir and delivering water from the reservoir to the electrolytic cell.

4. The method of claim 2 wherein the water is re-circulated through the electrolytic cell for a predetermined period of time and wherein when the predetermined period expires, the treated water is delivered across the surface.

5. The method of claim 4 further comprising providing a timer to automatically open a valve and deliver treated water across the surface when the predetermined re-circulation period expires.

6. The method of claim 1 further comprising inducing low pressure transients in the treated water as it is passing across the surface.

7. The method of claim 6 wherein the low pressure transients are induced by cycling a valve upstream of the surface.

8. The method of claim 6 wherein the low pressure transients are induced by cycling a valve to divert water away from the surface to a reservoir.

9. The method of claim 1 wherein the bacteria includes bacterial populations, mold and yeast colonies and the bacteria has at least a 4 Log reduction after passing the treated water over the surface.

10. The method of claim 1 wherein bacteria includes mold on at least a portion of the surface and cleaning comprises removing the mold from the surface.

11. The method of claim 1 wherein bacteria includes yeast on at least a portion of the surface and cleaning comprises removing the yeast from the surface.

12. The method of claim 1 wherein bacteria includes polysaccharide on at least a portion of the surface and cleaning comprises removing the polysaccharide from the surface.

13. A method of cleaning a fluid line comprising:
circulating water to an electrolytic cell to elevate a concentration of dissolved oxygen in the treated water; and
dispensing the treated water to the fluid line to clean the fluid line.

14. The method of claim 13 wherein a microorganism layer coats at least a portion of the fluid line and cleaning the fluid line comprises removing at least a portion of the microorganism layer from the fluid line.

15. The method of claim 13 wherein a bacteria layer coats at least a portion of the fluid line and cleaning the fluid line comprises removing at least a portion of the bacteria layer from the fluid line.

16. The method of claim 13 wherein yeast coats at least a portion of the fluid line and cleaning the fluid line comprises removing at least a portion of the yeast from the fluid line.

17. The method of claim 13 wherein a polysaccharide layer coats at least a portion of the fluid line and cleaning the fluid line comprises removing at least a portion of the polysaccharide layer from the fluid line.

18. The method of claim 13 wherein the fluid line is a beverage supply line.

19. The method of claim 13 wherein the fluid line is a medical supply line.

20. The method of claim 13 wherein the fluid line is a dental rinse line.

21. The method of claim 13 wherein the fluid line is a condiment dispensing line.

22. The method of claim 13 further comprising adding sodium chloride to the water.

23. The method of claim 13 further comprising re-circulating the water through the electrolytic cell to further elevate the concentration of dissolved oxygen in the water before dispensing the water to the fluid line.

24. The method of claim 23 wherein re-circulating the water comprises transferring the water from the electrolytic cell to a reservoir and transferring the water from the reservoir to the electrolytic cell.

25. The method of claim 24 wherein the water is re-circulated for a predetermined period of time prior to dispensing the water to the fluid line.

26. The method of claim 25 wherein the predetermined period of time is about 15 minutes.

27. The method of claim 13 further comprising inducing low pressure transients in the water dispensed to the fluid line to cause dissolved oxygen to bubble out of the water within the fluid line.

28. The method of claim 27 wherein the low pressure transients are induced by cycling a valve.

29. A cleaning system for cleaning bacteria from within a fluid supply line comprising:
a fluid supply line;
an electrolytic cell fluidly coupled to the fluid supply line;
bypass piping fluidly coupled to the fluid supply line and an inlet port for the electrolytic cell for delivering water from the fluid supply line to the electrolytic cell;
an outlet port fluidly connected to the electrolytic cell, the outlet port being configured to be coupled to the fluid supply line for dispensing water from the electrolytic cell to the fluid supply line for cleaning the fluid line;
an outlet valve fluidly coupled to the electrolytic cell between the electrolytic cell and the outlet port, for controlling water flow from the electrolytic cell to the outlet port;
a pre-charge valve fluidly coupled to the electrolytic cell and the bypass piping for controlling water flow from the bypass piping to the electrolytic cell; and
a flow activated switch coupled to the electrolytic cell, the flow activated switch being configured to close a power supply circuit path to the electrolytic cell when water flow is established through the electrolytic cell.

30. The system of claim 29 further comprising a reservoir positioned in-line between the bypass piping the inlet port for the electrolytic cell, the reservoir fluidly coupled to the bypass piping and the inlet port.
